(19) United States
(12) Patent Application Publication
(10) Pub. No.: US 2024/0425801 A1
(43) Pub. Date: Dec. 26, 2024

(54) COMPOSITION FOR PROTECTING A MICROORGANISM IN AN ACIDIC ENVIRONMENT

(71) Applicant: SETALG, DINARD (FR)

(72) Inventor: Sarah KUYLLE, Saint-Malo (FR)

(73) Assignee: SETALG, DINARD (FR)

(21) Appl. No.: 18/294,834

(22) PCT Filed: Aug. 2, 2022
(86) PCT No.: PCT/FR2022/051544

§ 371 Date: Feb. 2, 2024

(30) Foreign Application Priority Data
Aug. 3, 2021 (FR) 2108424

(51) Int. Cl. C12N 1/04; A23L 29/00; A23L 29/212; A23L 33/135; C12N 1/16; C12N 1/20; C12R 1/225

(52) U.S. Cl. CPC C12N 1/04; A23L 29/015; A23L 29/212; A23L 33/135; C12N 1/16; C12N 1/20; C12R 2001/225

(57) ABSTRACT

The invention relates to a composition comprising a calcium carbonate source, pregelatinized starch and a microorganism, a method for preparing a composition according to the invention, comprising a step of mixing a microorganism with a calcium carbonate source and pregelatinized starch, and using a mixture of pregelatinized starch and a calcium carbonate source for protecting a microorganism in an acidic environment.

### TECHNICAL FIELD

The invention relates to a composition comprising a calcium carbonate source, pregelatinized starch and a microorganism, a method for preparing a composition according to the invention, comprising a step of mixing a microorganism with a calcium carbonate source and pregelatinized starch, and using a mixture of pregelatinized starch and a calcium carbonate source for protecting a microorganism in an acidic environment.

### PRIOR ART

Microorganisms are increasingly used in many fields, notably in human and animal health (nutraceuticals) and plant health (agriculture).

In the nutraceutical sector, microorganisms are increasingly used for their probiotic characteristics.

Soils in agriculture are dynamic systems that contain a wide variety of microorganisms. However, a number of factors, such as the agricultural techniques used in recent decades and climate change, have upset pre-existing balances. For example, the use of large quantities of chemical inputs, cultivation techniques, etc. has led to a rarefaction, or even elimination, of some microorganisms from most cultivated soils, resulting in a loss of soil productivity.

To exert their beneficial effects, microorganisms must remain functional right up to their site of action, for example in the gastrointestinal tract in animal or human health, or in the soil in plant health. However, these microorganisms are fragile, as they are highly sensitive to environmental stresses, particularly pH variations.

In the nutraceutical field, the gastric compartment, due to its highly acidic pH, is known to degrade microorganisms of probiotic interest that are ingested by humans or animals.

As a result of soil acidification, microorganisms used in agriculture are also exposed to the same types of stress as those encountered in the nutraceutical field, which impedes their growth, stability and/or alters their effects. The low survival rate of microorganisms after their incorporation into the soil is one of the major factors limiting their effectiveness.

There is therefore a need to develop new strategies for protecting microorganisms against acidic pH.

In this context, the applicant has demonstrated, forming the basis of the present invention, that the use of a mixture of pregelatinized starch and a calcium carbonate source makes it possible to protect microorganisms against acid stress.

### SUMMARY OF THE INVENTION

The present invention, having applications in the nutraceutical and agricultural fields, is intended to provide a composition for protecting a microorganism against acid stress.

According to a first aspect, the invention relates to a composition comprising a calcium carbonate source, pregelatinized starch and a microorganism.

According to a second aspect, the invention relates to a method for preparing a composition according to the invention, comprising a step of mixing a microorganism with a calcium carbonate source and pregelatinized starch.

According to a third aspect, the invention relates to the use of a mixture of pregelatinized starch and a calcium carbonate source for protecting a microorganism in an acidic environment.

### DETAILED DESCRIPTION

### Definitions

The terms “pregelatinized starch” and “precooked starch” are used interchangeably to designate any native starch that has undergone heat treatment in the presence of water, so that it completely loses its granular structure and becomes soluble in cold water. Thus, by pregelatinized starch or precooked starch, we mean a state in which the starch is no longer in a granular state, i.e., in a state in which it is no longer in a semi-crystalline granular state characteristic of the state in which it is naturally present in the reserve organs and tissues of higher plants, in particular in cereal seeds, legume seeds, potato or manioc tubers, roots, bulbs, stems and fruits. This semi-crystalline state is essentially due to macromolecules of amylopectin, one of the two main constituents of starch. In their native state, starch grains have a degree of crystallinity ranging from 15 to 45%, depending essentially on their botanical origin and any processing they may have undergone. Placed under polarized light, granular starch shows a characteristic black cross under microscopy, known as a “Maltese cross”. This phenomenon of positive birefringence is due to the semi-crystalline organization of these granules: the average orientation of the polymer chains is radial. For a more detailed description of granular starch, reference may be made to chapter II entitled “Structure et morphologie du grain d'amidon” by S. Perez, in “Initiation à la chimie et à la physico-chimie macromoléculaires”, First Edition, 2000, Volume 13, pages 41 to 86, Groupe Français d'Etudes et d'Applications des Polymères [1].

According to the present invention, the starch used for preparing said pregelatinized starch is advantageously a native starch, and has therefore not undergone any prior treatment or modification. Alternatively, the starch used for preparing of said pregelatinized starch may be a modified starch having undergone prior treatment or modification, for example chemical modification such as cross-linking.

The pregelatinized state of the starch is obtained by cooking granular starch, incorporating water and applying thermal and mechanical energy. The breakdown of the semi-crystalline granular state of starch leads to amorphous pregelatinized starches with disappearance of the polarization Maltese cross. In the present invention, the pregelatinized starch may have a crystallinity level of less than 15%, preferably less than 5% and even more preferably less than 1%, i.e., in an essentially amorphous state.

In particular, this crystallinity level can be measured by X-ray diffraction, as described in patent U.S. Pat. No. 5,362,777 (column 9, lines 8 to 24).

According to a preferred mode of the present invention, the pregelatinized starch is advantageously substantially devoid of starch grains displaying, under polarized light microscopy, a Maltese cross, a sign indicative of the presence of semi-crystalline granular starch.

Pregelatinized starches according to the present invention can be obtained by hydrothermal gelatinization treatment of native starches, in particular by steam cooking, jet-cooker cooking, drum cooking, cooking in mixer/extruder systems followed by drying, for example in an oven, by hot air on a fluidized bed, on a rotating drum, by atomization, by extrusion or by freeze-drying. Such starches generally have a solubility in demineralized water at 20° C. greater than 5%, and more generally between 10 and 100%, and a starch crystallinity level of less than 15%, generally less than 5%, and more often less than 1%, or even zero. Examples include products manufactured and marketed by Roquette under the brand name PREGEFLO®.

The starch selected for preparing the native pregelatinized starch can be of any botanical origin that does not contain gluten, or whose gluten content does not exceed 20 mg/kg. Thus, starches derived from wheat (or wheat or spelt), barley, rye or triticale (wheat+rye) are generally to be banished as they contain gluten, unless their preparation methods have totally eliminated the gluten. In fact, there are wheat starches that are guaranteed gluten-free, obtained by a very specific method. Preferably, the native pregelatinized starch is prepared from a botanical source that does not contain gluten in its base. These may include starch from cereals such as corn, millet, buckwheat, oats, tapioca, sorghum or rice, tubers such as potatoes or manioc, or legumes such as peas and soybeans, starches rich in amylose or, conversely, rich in amylopectin (waxy), derived from these plants, and any mixtures of the aforementioned starches.

“Calcium carbonate” or “CaCO₃” is composed of carbonate ions (CO₃²⁻) and calcium ions (Ca²⁺). Calcium carbonate is the main component of limestones such as chalk, but also the main component of marble. It is also the main constituent of the shells of marine animals, coral and snails, as well as the eggshells of amniotes (except for therian mammals, whose internal eggs are shell-less). In the context of the present invention, the calcium carbonate source is selected from limestones (e.g., chalk), snail shells, eggshells, marine animal shells, corals and algae of the order Corallinales (e.g., lithothamnion). In a particularly preferred embodiment of the present invention, the calcium carbonate source is lithothamnion.

The term “lithothamnium” refers to a genus of red algae belonging to the Corallinaceae family, which has the ability to crystallize minerals and trace elements from the sea. It grows mainly in the North Atlantic Ocean, particularly on the seabed (up to 28 meters), where currents are less strong. Lithothamnion comprises 25 species. Lithothamnion is known for its high calcium carbonate content. Lithothamnion is used in nutraceuticals and agriculture, as well as in cosmetics, medicine and water treatment.

The term “microorganisms” refers to microscopic organisms such as bacteria, microscopic fungi (e.g., filamentous fungi) and yeasts. The microorganism may be viable and/or non-viable. By “non-viable” microorganism (e.g., non-viable probiotic bacteria), is meant a microorganism that is not capable of multiplying under any of the known growth conditions. By “viable” microorganism (e.g., a viable probiotic bacterium) is meant a microorganism capable of multiplying under appropriate conditions in which microorganism multiplication is possible. A microorganism that does not meet the definition of “non-viable” (as indicated above) is considered “viable”. On the other hand, a population of microorganisms of which only a portion (e.g., 10% or less) is still able to multiply under appropriate growth conditions, falls within the scope of the term “viable”. In the context of the present invention, the microorganism may be a microorganism of probiotic interest or a microorganism of agronomic interest.

A “microorganism of probiotic interest” (also referred to as “probiotic” in this description) refers to a viable or non-viable microorganism with a beneficial effect on health in humans or animals. The beneficial effect may be, for example, maintaining or improving the balance of the intestinal microbiota, decomposing (or fermenting) undigested food (e.g., dietary fiber) in the upper digestive tract, synthesizing vitamins, preventing the proliferation of pathogenic bacteria, boosting the immune system, and/or preventing gastrointestinal infections caused by antibiotic-resistant bacteria. Various microorganisms, such as yeasts, bacteria and in particular bifidobacteria, lactobacilli, leuconostocci, pediococci and lactococci, can be of probiotic interest. In food and feed, microorganisms of probiotic interest can be offered as dietary supplements.

A “microorganism of agronomic interest” is a living microorganism that has a beneficial effect on a plant. The beneficial effect may be, for example, the supply of nutrients useful for plant growth, such as atmospheric nitrogen fixation by the microorganism. Various microorganisms, such as bacteria, yeasts and microscopic fungi, e.g., filamentous microscopic fungi, can be of agronomic interest. In the context of the present invention, microorganisms can be selected from (i) atmospheric nitrogen-fixing bacteria, such as Azotobacter or Azospirillum (ii) plant Growth Promoting Rhizobacteria (PGPR), (iii) phosphorus-solubilizing bacteria such as Bacillus amyloliquefaciens, (iv) phytoprotective root bacteria (PGPR) capable of opposing the activity of pathogens such as Bacillus subtilis or Pseudomonas spp., (v) phytohormone-producing bacteria such as Bacillus amyloliquefaciens or Bacillus radicola, (vi) bacteria involved in the mineralization of organic matter such as Lactobacillus rhamnosus or Lactobacillus faciminis, (vii) iron-solubilizing bacteria such as Pseudomonas spp, (viii) silica-solubilizing bacteria, (ix) sulfur-oxidizing bacteria, (x) lactic acid bacteria such as Lactobacillus spp, Lactococcus spp. and Bifidobacterium spp. (xi) bacteria of the genus Enterococcus spp. and (xii) bacteria of the genus Pediococcus spp, (xiii) bacteria of the genus Bacillus licheniformis, (xiv) mycorrhizal fungi such as Rhizophagus irregularis, (xv) yeasts of the genus Saccharomyces cerevisiae and (xvi) a mixture of at least two micro-organisms selected from (i) to (xv).

The term “nutraceutical composition” refers to a composition intended for human or animal consumption which is capable of improving the state of well-being, performance and/or health of humans or animals.

The term “agronomic composition” refers to a composition intended for agriculture which has a beneficial effect on a plant.

The term “oral administration” refers to administration by ingestion, via the gastrointestinal tract.

The term “fertilizing substance” refers to a fertilizer and/or amendment.

The term “fertilizer” refers to fertilizing materials whose main function is to provide plants with elements directly useful for their nutrition (major fertilizing elements, secondary fertilizing elements and trace elements).

The term “amendment” refers to a substance designed to improve soil quality, and in particular to improve soil pH. Advantageously, the amendment is selected from basic mineral amendments of the limestone and/or limestones and magnesium type; humus-bearing amendments of the compost or manure type.

The water activity (symbol “a_{w}”) represents the water vapour pressure p of a moist product divided by the saturation vapour pressure p0 at the same temperature. This parameter reflects the interactions of water with the matrix of a composition. Microorganisms need free water (free for biochemical reactions) to grow. The higher the water activity, the greater the quantity of free water (1 being the maximum), and the more microorganisms will grow. The water activity can be reduced, for example, by drying or by adding a solute that will bind the water and render it unusable by microorganisms.

Particle size distribution reflects the size of the particles contained in a sample. The median size value (or “Dv(50)”) indicates that half the sample volume contains particles smaller than this value, and the other half contains particles larger than this value. Similarly, the values Dv(10) and Dv(90) indicate that respectively 10% and 90% of the sample volume contains particles smaller than this value. Dynamic light scattering (DLS) and laser diffraction are commonly used techniques for measuring the particle size distribution of a sample. DLS is particularly popular.

The present invention arises from the surprising advantages highlighted by the inventor of the effect of a mixture of a calcium carbonate source and pregelatinized starch in protecting a microorganism in an acidic environment.

### Composition

According to a first aspect, the invention relates to a composition comprising a calcium carbonate source, pregelatinized starch and a microorganism.

The applicant has shown that the microorganism is particularly well protected within the composition according to the invention, especially in acidic environments, such as gastric juice or acidic soils.

The water activity (a_{w}) of the composition according to the invention is preferably very low, so that the composition contains very little free water, which ensures good preservation of the microorganism prior to use of the composition. Thus, the water activity (a_{w}) of the composition according to the invention is preferably less than 0.1, preferably less than 0.06. A particularly preferred water activity (a_{w}) ranges from 0.005 to 0.1, for example from 0.01 to 0.1, for example from 0.02 to 0.06, for example about 0.05.

The calcium carbonate source raises the pH of the acidic environment in which it is found. The applicant has shown that this property protects microorganisms from the negative effects of acidity on their structure and function.

The calcium carbonate source can be selected from limestones (e.g., chalk), snail shells, eggshells, marine animal shells, corals and algae of the order Corallinales (e.g., lithothamnion). A particularly preferred calcium carbonate source is lithothamnion. Lithothamnion is sold commercially in various forms in the market, for example in form of powder.

Lithothamnium calcareum and/or Lithothamnium corallioides are the lithothamniums most commonly used in industry, and therefore preferred for the purposes of the present invention. Lithothamnium in form of is particularly suitable for a nutraceutical composition. A lithothamnium in form of powder particularly suitable for implementing the present invention is sold under the reference Algalithe® by the Setalg company (product made from Lithothamnium calcareum). Lithothamnium can, for example, be obtained by drying and then crushing lithothamnium harvested alive from the sea, or it can be obtained by drying and then crushing dead lithothamnium collected from beaches at low tide or from recesses close to the coast. As lithothamnion calcifies over time and forms sedimentary beds, these fossilized seaweeds are carried by the tides and accumulate in coastal hollows. In particular, they are harvested from well-defined plots of land in these recesses, in order to respect the renewal of the ecosystem.

Lithothamnion is in a form suitable for the intended application of the composition according to the invention. Preferably in form of powder or granules. According to one embodiment, the lithothamnion used in the present invention is obtained by drying and then grinding lithothamnion harvested alive from the sea. According to another embodiment, the lithothamnion used in the present invention is obtained by drying then crushing dead lithothamnion collected on beaches at low tide or in recesses close to the coast.

The calcium carbonate source can come in a variety of forms, such as in form of powder or in form of granules. The choice of form will depend in particular on the intended use of the composition. When the calcium carbonate source is in form of powder, it has a particle size distribution:
a Dv(90) ranging from 29 μm to 750 μm, for example from 50 μm to 500 μm, for example from 50 μm to 300 μm, for example from 75 μm to 250 μm, preferably from 90 μm to 200 μm; and/or
a Dv(50) ranging from 7 μm to 500 μm, e.g. 7 μm to 250 μm, e.g. 7 μm to 150 μm, e.g. 10 μm to 100 μm, e.g. 10 μm to 50 μm, e.g. 10 μm to 30 μm, preferably 15 μm to 25 μm; and/or
a Dv(10) ranging from 1 μm to 270 μm, e.g. from 1 μm to 100 μm, e.g. from 1 μm to 50 μm, e.g. from 1 μm to 25 μm, e.g. from 1 μm to 10 μm, preferably from 1 μm to 5 μm.

The amount of the calcium carbonate source in the composition of the invention can range from 10% to 95% by weight relative to the total weight of the composition, preferably from 25% to 85%, more preferably from 30% to 80%, more preferably from 40% to 65%, more preferably from 50% to 65%. The amount of the calcium carbonate source in the composition may vary, for example, according to the intended use of the composition or the nature of the calcium carbonate source.

Pregelatinized starch has the property of forming a gel at acidic pH. The applicant has demonstrated that it amplifies the protective effect observed on microorganisms when combined with calcium carbonate.

The pregelatinized starch is preferably prepared from a starch-containing plant source, preferably selected from pregelatinized corn starch, pregelatinized pea starch, pregelatinized potato starch, pregelatinized tapioca starch, pregelatinized rice starch, pregelatinized cassava starch. In a particularly preferred embodiment, the composition according to the invention contains a pregelatinized corn starch or a pregelatinized potato starch.

Pregelatinized starch can be in various forms, for example in form of powder or in form of granules. The choice of the form will depend in particular on the intended use of the composition. When the pregelatinized starch source is in powder form, it has a particle size distribution:
a Dv(90) ranging from 90 μm to 1300 μm, for example ranging from 90 μm to 500 μm, for example ranging from 90 μm to 250 μm, preferably ranging from 90 μm to 150 μm; and/or
a Dv(50) ranging from 40 μm to 500 μm, for example ranging from 40 μm to 250 μm, for example ranging from 40 μm to 100 μm, preferably ranging from 40 μm to 60 μm; and/or
a Dv(10) ranging from 10 μm to 150 μm, for example ranging from 10 μm to 100 μm, for example ranging from 10 μm to 50 μm, preferably ranging from 10 μm to 20 μm.

The amount of pregelatinized starch in the composition of the invention can range from 10% to 90% by weight relative to the total weight of the composition, preferably from 10% to 70%, more preferably from 25% to 50%. The amount of pregelatinized starch in the composition may also vary, for example, according to the intended use of the composition or the nature of the pregelatinized starch.

In a preferred embodiment of the composition according to the invention, the ratio [mass of calcium carbonate source]: [mass of pregelatinized starch] ranges from 0.4 to 5.7, for example from 0.4 to 3, for example from 0.5 to 2, for example from 0.8 to 1.9, preferably equal to 1 +/−0.1.

The microorganism is selected according to the intended use of the composition according to the invention. Thus, the microorganism may, for example, be a microorganism of probiotic interest or a microorganism of agronomic interest. For a nutraceutical composition according to the invention, the microorganism is preferably a microorganism of probiotic interest, for example a probiotic bacterium.

The probiotic bacteria referred to in the present invention may be any of the probiotic bacteria known and available from commercial and/or public sources, such as the Collection Nationale de Cultures de Microorganismes (CNCM), the American Tissue Culture Collection (ATCC), the Belgian Coordinated Collections of Microorganisms/Laboratorium voor Microbiologie Universiteit Gent (BCCM/LMG) or others.

In particular, probiotic bacteria suitable for the invention may be selected from the group comprising probiotic bacteria of the lactic ferment type, selected from bacteria of the genera Lactobacillus spp., Bifidobacterium spp. and mixtures thereof.

Non-limiting examples of bacteria of the Lactobacillus spp. genus include: Lactobacillus acidophilus, Lactobacillus brevis, Lactobacillus casei, Lactobacillus crispatus, Lactobacillus curvatus, Lactobacillus delbrueckii, Lactobacillus farciminis, Lactobacillus fermentum, Lactobacillus gasseri, Lactobacillus helveticus, Lactobacillus johnsonii, Lactobacillus paracasei, Lactobacillus plantarum, Lactobacillus reuteri, Lactobacillus rhamnosus.

Non-limiting examples of bacteria of the Bifidobacterium spp. genus include: Bifidobacterium adolescentis, Bifidobacterium bifidum, Bifidobacterium breve, Bifidobacterium infantis, Bifidobacterium animalis, Bifidobacterium longum, Bifidobacterium thermophilum .

According to one embodiment, a probiotic bacterium suitable for the invention is selected from Lactobacillus rhamnosus, Bifidobacterium animalis subsp. lactis, and combinations thereof.

As an example, one or more of the following probiotic strains may be used:
Lactobacillus rhamnosus HN001™ marketed by Dupont;
Bifidobacterium animalis subsp. lactis BB-12® marketed by Chr. Hansen;
Lactobacillus plantarum (LMG P-21021) marketed under the name LP01 by Probiotical® S.p.A., based in Italy; registered in 2001 with BCCM/LMG (Belgian Coordinated Collections of Microorganisms/Laboratorium voor Microbiologie Universiteit Gent).
Lactobacillus gasseri (LMG 26661) marketed under the name THT 031301 by THT s.a. (Belgium) and registered with BCCM/LMG in 2011;
Lactobacillus gasseri SGL09 marketed by Nutraceutica® S.r.l. in Italy.

Other suitable probiotic bacteria are also available, such as those described in EP1945235B1 and WO2009014421A1.

It should be noted that the Lactobacillus rhamnosus and Bifidobacterium animalis subsp. lactis species are naturally present in the digestive tracts of humans and animals.

The probiotic bacteria used in the present invention can be produced using any standard fermentation process known in the prior art. They can be in freeze-dried form, particularly presented in form of powder.

The probiotic bacteria used in the present invention may be viable or non-viable. The use of viable probiotic bacteria offers the advantage that these bacteria can form part of the intestinal microbiota, thus providing additional health benefits. The viability of the probiotic bacteria present in the composition of the invention can be assessed by any conventional bacterial counting technique known to those skilled in the art, for example by the Colony Forming Unit (CFU) method after decimal dilutions and spreading on Petri dishes.

The number of microorganisms must be sufficient to have the desired effect, which depends on the intended use of the composition according to the invention. Preferably, the number of microorganisms ranges from 10³ to 10¹³ cells/g of composition (or CFU for “Colony Forming Unit”, when the microorganism is viable), preferably from 10⁵ to 10¹² cells/g of composition, for example from 10⁵ to 10¹¹ cells/g of composition. The number of microorganisms in the composition may vary, for example, according to the intended use of the composition or the nature of the microorganism.

The composition according to the invention can be encapsulated in one or more suitable encapsulation materials, which makes it possible in particular to protect the microorganism from oxygen and moisture, for example, and to facilitate handling of the composition according to the present invention and ensure preservation of the microorganism in the composition according to the invention during storage.

As non-limiting examples of suitable encapsulation materials, mention may be made of fatty acids and waxes, monoglycerides of saturated fatty acids, diglycerides of saturated fatty acids, polyglycerols esterified with saturated fatty acids, free saturated fatty acids, glyceryl dipalmitostearate, beepolle and clay-based complexes, or others (see for example patent applications WO 01/68808 (LALLEMAND S. A.), WO2013114185A1 (Probiotical S.P.A.), and WO 2013/153117 (Laboratoire BEEPRATTE)). Suitable encapsulation materials can be gastroresistant to protect the microorganism from gastric fluids during its gastrointestinal journey (resistance to gastric acidity, for example) for improved activity in the host's intestine or colon.

As previously explained, the composition according to the invention can be used in different fields, such as human or animal nutrition, in which case it is referred to as a nutraceutical composition, or in agriculture, in which case it is referred to as a composition of agronomic interest.

The skilled in the art will have no difficulty in adapting the composition to the intended use. For example, the composition according to the invention may be in form of powder or in form of granules. The form of powder is particularly suitable for a nutraceutical composition, since it must be suitable for oral administration. Nevertheless, it is entirely possible to have a nutraceutical composition in form of granules suitable for oral administration, particularly in animal feed. A composition in form of granules is particularly suitable for a composition of agronomic interest, as this form facilitates spreading over large areas of soil. Nevertheless, it is perfectly possible to have a composition of agronomic interest in form of powder, particularly when the composition does not need to be applied to large areas of soil.

Similarly, the microorganism will be selected according to the intended use. For example, a nutraceutical composition will contain one or more microorganisms of probiotic interest and a composition of agronomic interest will contain one or more microorganism(s) of agronomic interest.

When the composition is a nutraceutical composition, it may also comprise, in addition to the microorganism, calcium carbonate and pregelatinized starch, one or more additional active ingredients. Naturally, these skilled in the art will take care to choose this or these possible additional active ingredients, and/or their quantity, in such a way that the advantageous properties of the composition according to the invention are not, or substantially not, altered. Preferably, the additional active ingredient(s) is/are selected from dried plant and/or fruit extracts, vitamins, amino acids, mineral salts, trace elements and mixtures thereof. A nutraceutical composition according to the invention may also comprise suitable excipients such as gelatin, modified starch, vegetable gums, maltodextrins, alginates, dextran, milk powder, magnesium stearate, or others. Magnesium stearate has the property of improving the flowability of the composition, making it easier, for example, to prepare capsules which comprise the composition according to the invention.

In a particular embodiment, the nutraceutical composition consists essentially of a calcium carbonate source, pregelatinized starch and a microorganism.

When the composition is a composition of agronomic interest, it may also comprise, in addition to the microorganism, calcium carbonate and pregelatinized starch, one or more additional compounds. Naturally, the skilled in the art will take care to choose this or these possible additional compounds, and/or their quantity, in such a way that the advantageous properties of the composition according to the invention are not, or substantially not, altered. Preferably, the additional compound(s) are selected from fertilizing substances, such as fertilizers or soil amendments.

The fertilizer can be one or more active substances selected from nitrogen, phosphorus, potassium, urea, ammonium sulfate, ammonium nitrate, phosphate, potassium chloride, ammonium sulfate, magnesium nitrate, manganese nitrate, zinc nitrate, copper nitrate, phosphoric acid, potassium nitrate, boric acid and mixtures thereof, preferably a mixture of nitrogen, potassium and phosphorus or a mixture of phosphorus and potassium. The amendment may be one or more active substances selected from basic mineral amendments of the limestone type, basic mineral amendments of the magnesium type, humus-containing amendments of the compost type and/or humus-containing soil amendments of the manure type.

### Method and Use

According to a second object, the invention relates to a method for preparing a composition according to the invention, comprising a step of mixing a microorganism with a calcium carbonate source and pregelatinized starch.

The preparation of a composition according to the invention is carried out according to the conventional methods described in the literature. Those skilled in the art will have no difficulty in carrying out the mixtureing, for example by mixing powders and/or granules.

According to a third object, the invention relates to the use of a mixture of pregelatinized starch and a calcium carbonate source for protecting a microorganism in an acidic environment. The applicant has found that a mixture of pregelatinized starch and a calcium carbonate source can be used to protect a microorganism in an acidic environment, such as the upper part of the gastrointestinal tract or acidic soil.

In the context of the present invention, “protecting a microorganism in an acidic environment” refers to improving the survival rate of the microorganism in an acidic environment and/or improving the integrity of the microorganism's cell membrane in an acidic environment. Thus, the present invention also relates to the use of a mixture of pregelatinized starch and a calcium carbonate source to improve the survival rate of the microorganism in an acidic environment and/or to improve the integrity of the cell membrane of a microorganism in an acidic environment. The applicant found that the survival rate of microorganisms and/or the integrity of the cell membrane of microorganisms in an acidic environment were significantly improved when said microorganisms were mixed with pregelatinized starch and a calcium carbonate source, compared with microorganisms not mixed with pregelatinized starch and a calcium carbonate source.

The integrity of the cell membrane of microorganisms can be easily measured by techniques well described in the literature, such as flow cytometry. For example, two flow cytometry markers can be used, Syto®24 and propidium iodide, which are more or less permeable depending on the membrane state of the cells (see the method described in the Examples).

The survival rate of microorganisms is usually measured by determining the CFU survival rate (see method described in Examples).

Detailed characteristics in the “composition” section of the present description, particularly with regard to pregelatinized starch, the calcium carbonate source and the microorganism, apply to the method and use according to the present invention.

Particular embodiments of the present invention are illustrated in the following examples.

### DESCRIPTION OF THE FIGURES

FIG. 1: Diagram detailing the Gastro-Duodeno-Ileal Model (GDIM) used in the examples.

FIG. 2A: CFU survival rate of L. rhamnosus strain HN001™ at the end of GDIM with the use of native starch, lithothamnion or ground calcium carbonate, alone and in mixtures.

FIG. 2B: CFU survival rate of L. rhamnosus strain HN001™ at the end of GDIM with the use of pregelatinized corn starch, lithothamnion or ground calcium carbonate, alone and in mixtures.

FIG. 3: Schematic diagram of the flow cytometry (FMC) implemented in the examples.

FIG. 4: Cell population distribution of the L. rhamnosus HN001™ strain according to their membrane state, upstream and downstream of the GDIM, depending on the excipients/mixtures of excipients used.

FIG. 5: Survival rate of L. rhamnosus HN001™ strain CFUs at the end of GDIM with the use of pregelatinized starch and lithothamnion, alone and in mixtures at different ratios.

FIG. 6: CFU survival rate of L. rhamnosus strain HN001™ at the end of GDIM with the use of 50/50 mixtures of pregelatinized starches of different plant origins and lithothamnion.

FIG. 7A: CFU survival rate of the B. animalis subsp. lactis BB-12® strain at the end of GDIM with the use of pregelatinized corn starch alone or in a 50/50 mixture with lithothamnion.

FIG. 7B: CFU survival rate gain measured for L. rhamnosus HN001™ and B. animalis subsp. lactis BB-12® strains with a 50/50 lithothamn/pregelatinized corn starch mix compared with pregelatinized corn starch alone.

### EXAMPLES

### Example 1: Preparation of Test Formulations

The formulations tested were prepared by mixing the calcium carbonate source and pregelatinized starch, then mixeding with the microorganism. The mixture was then placed in size 0 HPMC capsules using a capsule filer.

The compositions of the formulations prepared are detailed in Table 1.

TABLE 1

| Formula | | Calcium Carbonate | |
|---|---|---|---|
| Reference | Microorganism | Source | Starch |
| HN001 - 100% | 1 gram of | — | 24 g native corn |
| native corn starch | Lactobacillus | | starch (corn starch |
| | rhamnosus HN001 ™ | | with 5% residual |
| | at 5.0.10¹¹ CFU/g | | moisture (Roquette) |
| HN001 - 50% native | 1 gram of | 12 g lithothamnion | 12 g native corn |
| corn starch/50% | Lactobacillus | (Algalithe ® | starch (corn starch |
| lithothamnion | rhamnosus HN001 ™ | Dv(90) <150 μm) | with 5% residual |
| | at 5.0.10¹¹ CFU/g | | moisture (Roquette) |
| HN001 - 100% | 1.1 gram of | — | 23.9 g pregelatinized |
| pregelatinized corn | Lactobacillus | | corn starch |
| starch | rhamnosus HN001 ™ | | (Prégéflo ® M) |
| | at 4.5.10¹¹ CFU/g | | |
| HN001 - 50% | 1.1 gram of | 11.95 g | 11.95 g |
| pregelatinized corn | Lactobacillus | lithothamnion | pregelatinized corn |
| starch/50% | rhamnosus HN001 ™ | (Algalithe ® | starch (Prégéflo ® M) |
| lithothamnion | at 4.5.10¹¹ CFU/g | Dv(90) <150 μm) | |
| HN001 - 100% | 1.1 gram of | 23.9 g lithothamnion | — |
| lithothamnion | Lactobacillus | (Algalithe ® | |
| | rhamnosus HN001 ™ | Dv(90) <150 μm) | |
| | at 4.5.10¹¹ CFU/g | | |
| HN001 - 100% | 1 gram of | 24 g ground calcium | — |
| ground calcium | Lactobacillus | carbonate | |
| carbonate | rhamnosus HN001 ™ | | |
| | at 5.0.10¹¹ CFU/g | | |
| HN001 - 50% native | 1 gram of | 12 g ground calcium | 12 g native corn |
| corn starch/50% | Lactobacillus | carbonate | starch |
| ground calcium | rhamnosus HN001 ™ | | |
| carbonate | at 5.0.10¹¹ CFU/g | | |
| HN001 - 50% | 1 gram of | 12 g ground calcium | 12 g pregelatinized |
| pregelatinized corn | Lactobacillus | carbonate | corn starch |
| starch/50% ground | rhamnosus HN001 ™ | | (Prégéflo ® M) |
| calcium carbonate | at 5.0.10¹¹ CFU/g | | |
| HN001 - 70% | 1.1 gram of | 7.2 g lithothamnion | 16.7 g pregelatinized |
| pregelatinized corn | Lactobacillus | (Algalithe ® | corn starch |
| starch/30% | rhamnosus HN001 ™ | Dv(90) <150 um) | (Prégéflo ® M) |
| lithothamnion | at 4.5.10¹¹ CFU/g | | |
| HN001 - 35% | 1.1 gram of | 15.5 g lithothamnion | 8.4 g pregelatinized |
| pregelatinized corn | Lactobacillus | (Algalithe ® | corn starch |
| starch/65% | rhamnosus HN001 ™ | Dv(90) <150 μm) | (Prégéflo ® M) |
| lithothamnion | at 4.5.10¹¹ CFU/g | | |
| HN001 - 15% | 1.1 gram of | 20.3 g lithothamnion | 3.6 g pregelatinized |
| pregelatinized corn | Lactobacillus | (Algalithe ® | corn starch |
| starch/85% | rhamnosus HN001 ™ | Dv(90) <150 μm) | (Prégéflo ® M) |
| lithothamnion | at 4.5.10¹¹ CFU/g | | |
| HN001 - 50% | 1 gram of | 12 g lithothamnion | 12 g pregelatinized |
| pregelatinized potato | Lactobacillus | (Algalithe ® | potato starch |
| starch/50% | rhamnosus HN001 ™ | Dv(90) <150 μm) | (Prégéflo ® P100G) |
| lithothamnion | at 5.0.1⁰¹¹ CFU/g | | |
| HN001 - 50% | 1 gram of | 12 g lithothamnion | 12 g pregelatinized |
| pregelatinized pea | Lactobacillus | (Algalithe ® | pea starch |
| starch/50% | rhamnosus HN001 ™ | Dv(90) <150 μm) | (Prégéflo ® L100G) |
| lithothamnion | at 5.0.10¹¹ CFU/g | | |
| Bb12 - 100% | 3 grams of | 12 g lithothamnion | — |
| lithothamnion | Bifidobacterium | (Algalithe ® | |
| | animalis subsp. lactis | Dv(90) <150 μm) | |
| | BB-12 ® at 1.5.10¹¹ | | |
| | CFU/g | | |
| Bb12 - 100% | 3 grams of | — | 12 g pregelatinized |
| pregelatinized corn | Bifidobacterium | | corn starch |
| starch | animalis subsp. lactis | | (Prégéflo ® M) |
| | BB-12 ® at 1.5.10¹¹ | | |
| | CFU/g | | |
| Bb12 - 50% | 3 grams of | 6 g lithothamnion | 6 g pregelatinized |
| pregelatinized corn | Bifidobacterium | (Algalithe ® | corn starch |
| starch/50% | animalis subsp. lactis | Dv(90) <150 μm) | (Prégéflo ® M) |
| lithothamnion | BB-12 ® at 1.5.10¹¹ | | |
| | CFU/g | | |

### Example 2: Gastro-Duodeno-Ileal Model (GDIM)

The Gastro-Duodeno-Ileal Model (GDIM) is a static model of human digestion in vitro. It mimics certain conditions in the upper part of the human digestive tract (pH, enzymes, etc.). The GDIM model simulates passage through 3 successive compartments:
The first compartment (which simulates the stomach) is made up of a Citrate/Sodium Phosphate buffer pH 3.0 supplemented with an electrolyte solution and 0.003% (w/w) pepsin.
The second compartment (which simulates the duodenal compartment) is created by adding to the first compartment a solution of Di-Sodium hydrogen phosphate dihydrate (HNa₂O₄P, 2H₂O) to achieve a pH of 6.5, bile at 0.3% (w/w) and trypsin at 0.007% (w/w).
The third compartment (which simulates the ileal compartment) is created by adding to the second compartment a solution of Di-Sodium hydrogen phosphate dihydrate (HNa₂O₄P, 2H₂O) to achieve a pH of 7.0.

The GDIM model is used to assess the ability of the compositions tested for protecting microorganisms in an acidic environment.

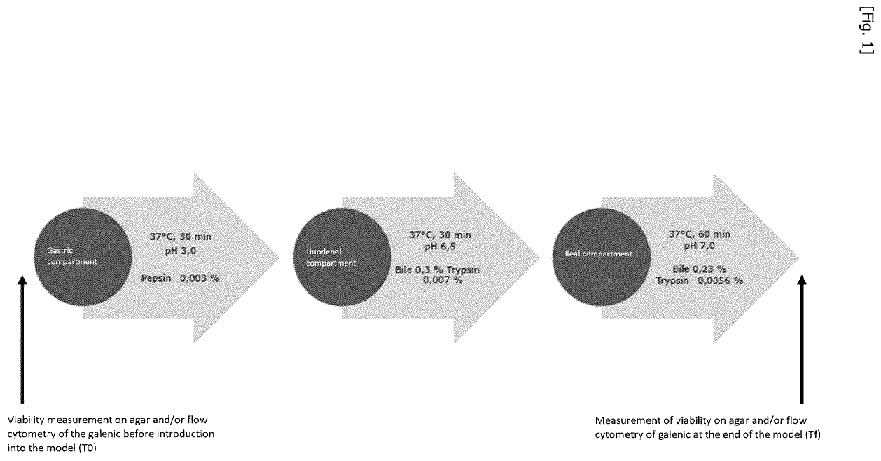

The GDIM is described in detail in reference [2] and illustrated in FIG. 1.

### 3.1 Preparation of Mixtures and Packaging in HPMC (HydroxyPropylMethylCellulose) Capsules

The microorganism/starch/calcium carbonate source mixtures were prepared to a target concentration of between 2.10¹⁰ CFU/g and 3.10¹⁰ CFU/g, then packaged in HPMC capsules according to the protocol detailed in Example 1.

### 3.2 Performing GDIM Tests

### Implementing the Model

All tests were performed in triplicate or more. At the beginning and end of each of the 3 GDIM compartments, PH levels were measured and visual observations were made.

4 ml of water were added to a sterile jar containing the capsule and its spiral support (to ensure proper immersion of the capsule in the model). The contents of the jar were then immediately poured into the first gastric compartment (flask). The flask was incubated for 30 min at 37° C. with orbital shaking.

Passage into the second compartment was achieved by adding to the flask a solution of Di-Sodium hydrogen phosphate dihydrate (HNa₂O₄P, 2H₂O) to achieve a pH of 6.5, 0.3% (w/w) bile and 0.007% (w/w) trypsin. The flask was incubated for 30 min at 37° C. with orbital shaking.

Passage into the ileal compartment was achieved by adding a solution of Di-Sodium hydrogen phosphate dihydrate (HNa₂O₄P, 2H₂O) to achieve a pH of 7.0. The flask was incubated for 60 min at 37° C. with orbital shaking. At the end of the model, the contents of the compartment were poured into a Stomacher bag, and the capsule was released from its spiral support and dissolved manually (if still present). After homogenization with the Stomacher, cascade dilutions to 1/10th were made in peptone water.

### Measuring Viabilities (CFU)

Viability measurements were carried out upstream and downstream of the GDIM model by inoculation with cysteine-supplemented MRS agar from several dilutions (minimum of 3 agars/dilution). Petri dishes were then incubated anaerobically at 37° C. for at least 48 h.

These viability measurements made it possible to measure the CFU survival rate of the microorganism for each of the formulations tested according to the following formula:

CFU survival rate (%)=(Number of CFUs after GDIM/Number of CFUs before GDIM)×100.

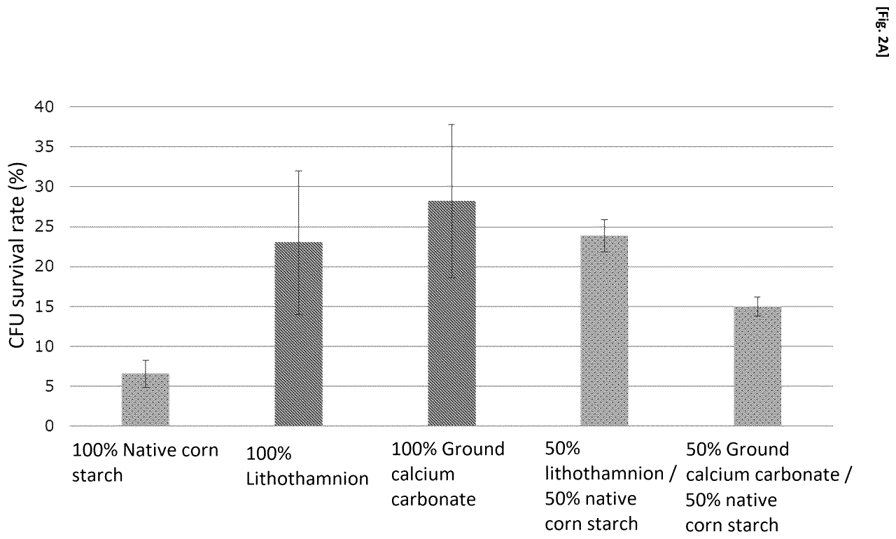

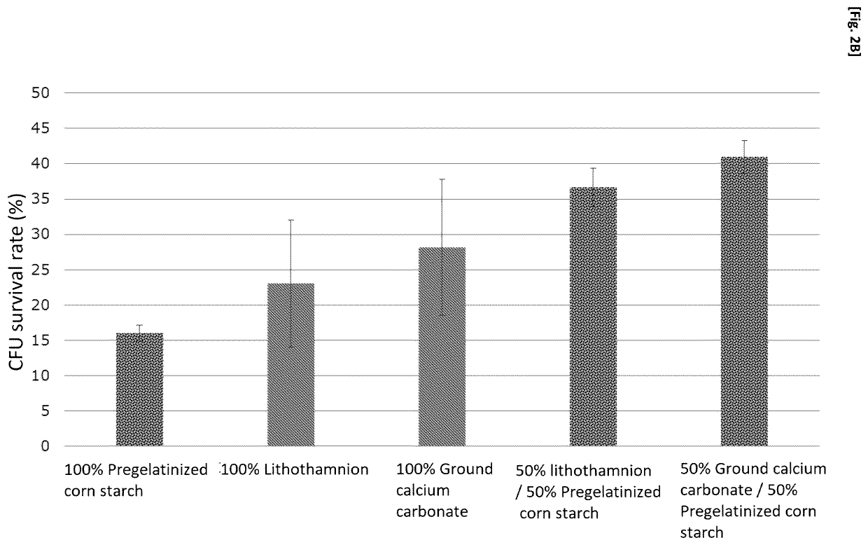

The results are shown in FIG. 2.

The results obtained with native corn starch (FIG. 2A) indicate that the survival rates obtained with the different calcium carbonate sources used alone are higher than those measured for native corn starch/calcium carbonate source mixtures, thus showing no synergistic effect. In contrast, the results obtained with pregelatinized corn starch (FIG. 2B) indicate that the survival rates obtained for the pregelatinized corn starch/calcium carbonate source mixtures are higher than those measured for each of the ingredients used separately, thus signifying the existence of a synergistic effect for bacterial survival.

### Measuring Membrane Integrity by Flow Cytometry

Flow cytometry analyses (FCA) were performed upstream and downstream of the GDIM model according to protocol B from ISO 19344 IDF 232 v2015, using the membrane integrity markers Syto®24 and propidium iodide.

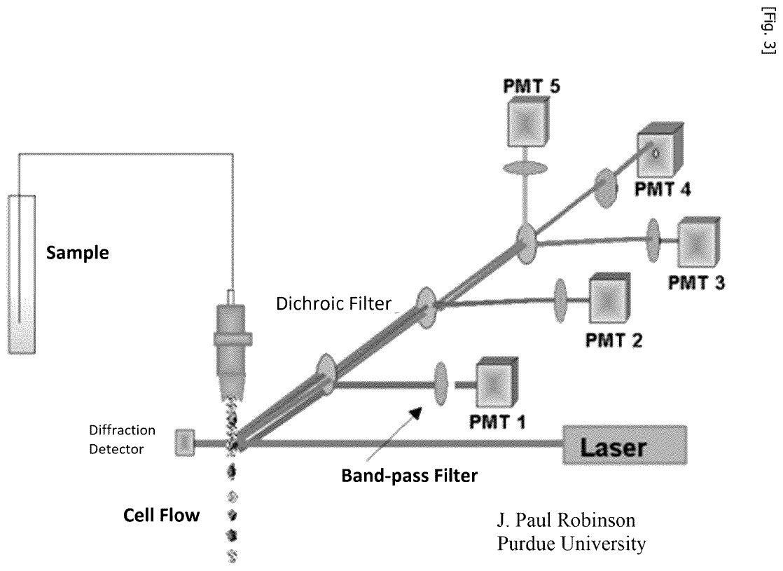

FCA is a technique for the individual, quantitative and qualitative characterization of particles suspended in a liquid. Cells are passed one by one through a light source. Scattered and emitted light is measured by a range of detectors. The measurements obtained are computer-processed to generate a multi-parameter data set. FCA is illustrated in FIG. 3.

Measurement of membrane integrity is based on the following principle:
Syto®24 penetrates the membranes of all cells and emits a green fluorescence upon binding to nucleic acids,
Propidium iodide only penetrates bacteria with damaged membranes and emits a red fluorescence when it binds to nucleic acids.

Thus, the duality between these 2 markers makes it possible to distinguish 3 cell populations according to their membrane state:
Bacteria with intact cell membranes emit green fluorescence (intact cells=IC).
Bacteria with minor membrane damage show both green and red fluorescence (damaged cells=CE).
Bacteria with severely damaged membranes emit a more intense red fluorescence (dead cells=CM).

These cytometric measurements can therefore be used to qualify and quantify the evolution of these populations upstream and downstream of the model.

Measurement of membrane integrity was carried out in parallel with the agar-based viability measurement described above. The dilution containing a concentration of approximately 107 cells/mL was used for labeling. 50 μL of this dilution was taken and placed in a microtube containing 440 μL of peptone water with Tween 80 added, to which 5 μL of 0.1 mM Syto®24 and 5 μL of 0.2 mM propidium iodide were added. After vortexing for at least 5 seconds, the mixture was incubated for 15 minutes at 37° C. in the dark. The reaction mixture was then vortexed again and immediately analyzed using a flow cytometer.

These cytometric measurements made it possible to measure the IC survival rate of the microorganism for each of the formulations tested according to the following formula:

IC survival rate (%)=(Number of IC after GDIM/Number of IC before GDIM)×100.

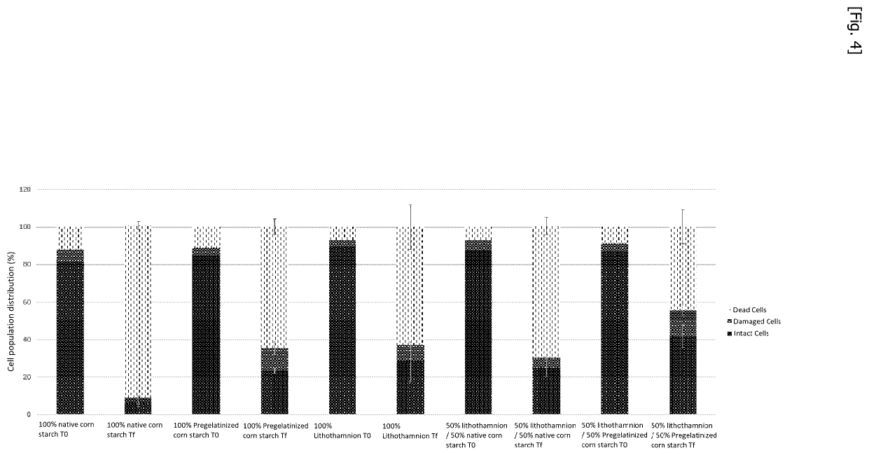

The results are shown in FIG. 4.

The results obtained upstream of the model show that, for each of the galenicals tested, between 82 and 90% of cells are considered to be intact. After passage through the in vitro digestion model, this population of intact cells drops to an average of 5, 16 and 25% for native corn starch, pregelatinized corn starch and lithothamnion, respectively. The use of the native corn starch/lithothamnion mixture did not improve cell integrity, since at the end of the model the rate of intact cells was 22% on average. In contrast, the use of the pregelatinized corn starch/lithothamnion mixture preserved 37 % of intact cells at the end of the model. The synergistic effect observed for CFUs in FIG. 2 is therefore also visible for cell integrity with the use of a mixture of a pregelatinized starch source and calcium carbonate.

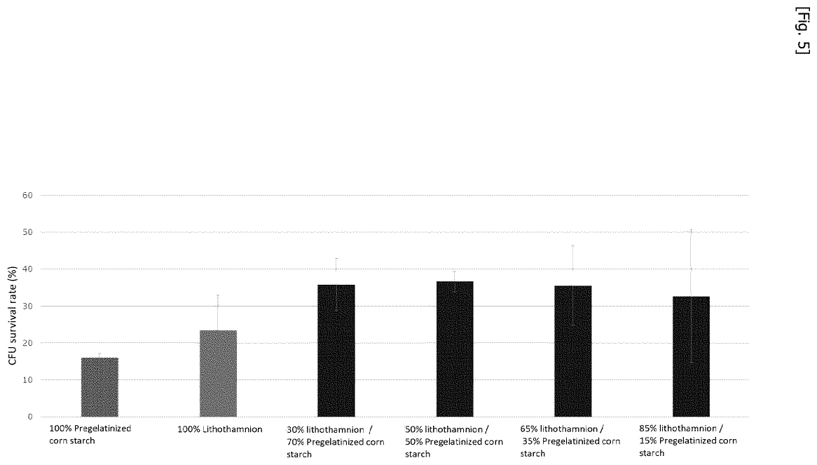

Results obtained with different ratios of lithothamnion to pregelatinized corn starch are shown in FIG. 5.

The results obtained indicate that the protective effect of the mixture of pregelatinized corn starch and lithothamnion is visible for the 4 ratios tested, with a slight decrease for the 85% lithothamnion/15% pregelatinized corn starch ratio. It may be noted that the standard deviations are greater when lithothamnion is used alone or with a high content in the mixture. This could be due to the greater variation in pH in the medium when the gel is absent (lithothamnion alone) or less stable (85% lithothamnion/15% pregelatinized corn starch).

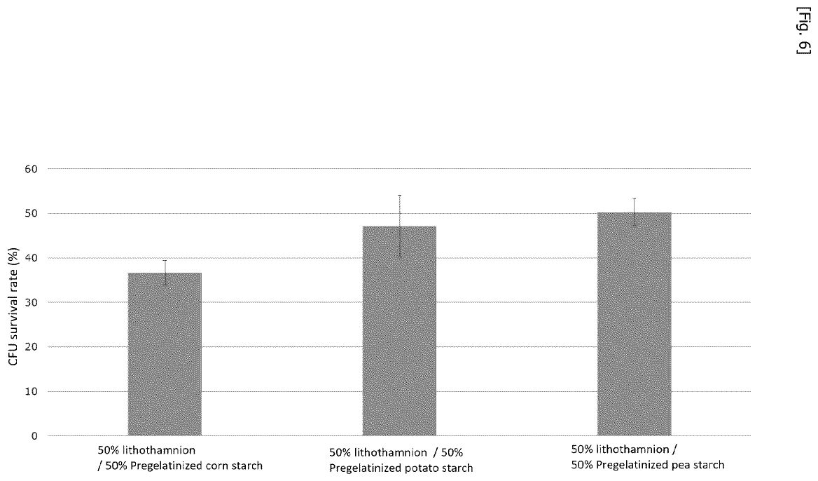

Results obtained with different sources of pregelatinized starch are shown in FIG. 6.

The results obtained indicate that the survival rates measured with 50/50 lithothamnion/pregelatinized potato starch and 50/50 lithothamnion/pregelatinized pea starch mixtures give higher survival rates than those already obtained with a 50/50 lithothamnion/pregelatinized corn starch mixture, confirming the protection that a lithothamnion/pregelatinized starch combination offers for the survival of microorganisms in acidic environments.

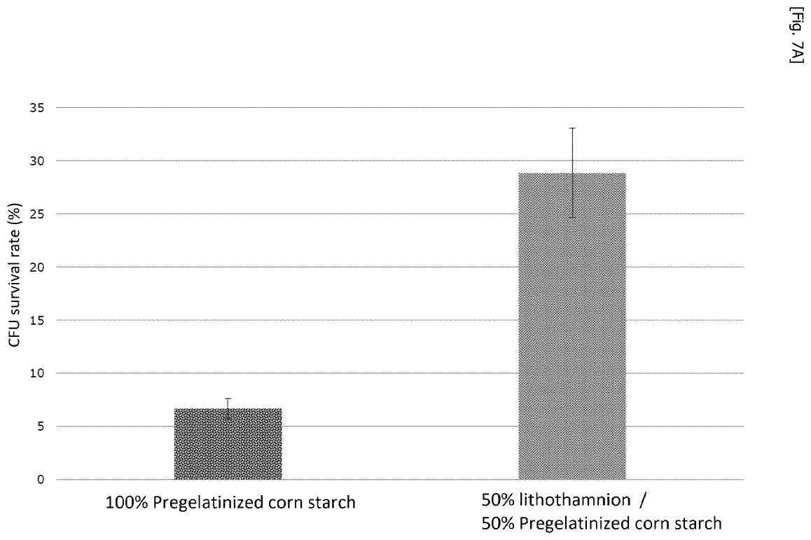

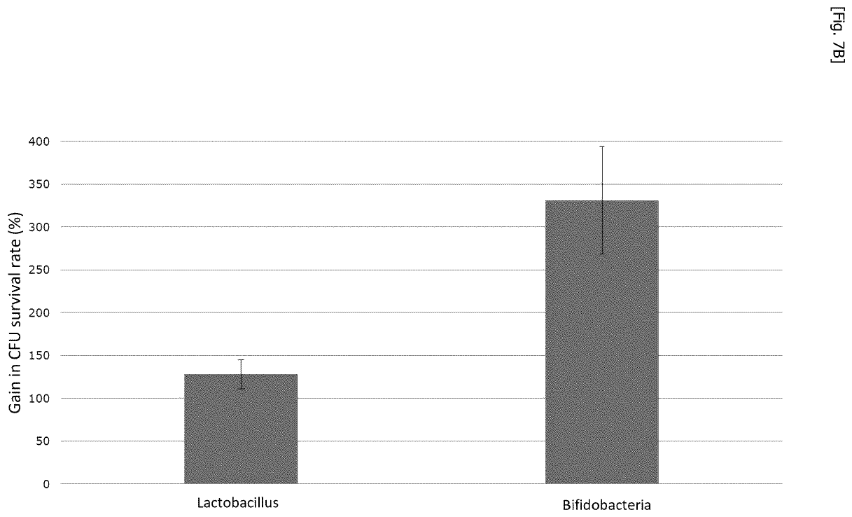

Results obtained with different bacterial groups are shown in FIG. 7.

The results show a significant improvement in the CFU survival rate of the B. animalis subsp. lactis BB-12® strain using the 50/50 lithothamnion/pregelatinized corn starch mixture, compared with the use of pregelatinized corn starch alone (FIG. 7A).

The gain in survival rate for each of the 2 strains (L. rhamnosus HN001™ and B. animalis subsp. lactis BB-12®) can be calculated as follows, in order to better visualize the protective effect of the calcium carbonate source/pregelatinized starch combination:

Survival rate gain (%)=((Mixture survival rate-pregelatinized starch alone survival rate)/pregelatinized starch alone survival rate)×100.

The results (FIG. 7B) show a substantial improvement in CFU survival for these 2 bacteria belonging to 2 different bacterial groups (Firmicutes and Actinobacteria). The improved survival of the B. animalis subsp. lactis BB-12® strain is all the more significant as this strain is known for its high sensitivity to acidic pH, compared to the L. rhamnosus HN001™ strain which is more robust towards this parameter (7% +/−1% and 16% +/−1% with 100% pregelatinized corn starch, respectively).

### REFERENCES

[1] S. Perez, in “Initiation à la chimie et à la physico-chimie macromoléculaires”, First Edition, 2000, Volume 13, pages 41 to 86, Groupe Français d'Etudes et d'Applications des Polymères.
[2] Kuylle et al (2016). Interest of the simplified in vitro gastro-duodeno-leal model (GDIM) to assess the performance of oral forms of probiotics. J. Int. Soc. Microbiota, vol.3, p.101, DOI: 10.18143/JISM_v3i1.

1. A composition comprising a calcium carbonate source, pregelatinized starch and a microorganism.

2. The composition according to claim 1, wherein the water activity (a_{w}) is less than 0.1.

3. The composition of any one of claim 1, wherein the calcium carbonate source is selected from limestones, snail shells, eggshells, marine animal shells, corals and algae of the order Corallinales.

4. The composition according to claim 1, wherein the amount of the calcium carbonate source ranges from 10% to 95% by weight of the total weight of the composition.

5. The composition of claim 1, wherein the calcium carbonate source has a particle size distribution Dv(90) ranging from 29 μm to 750 μm and/or a Dv(50) ranging from 7 μm to 500 μm and/or a Dv(10) ranging from 1 μm to 270 μm.

6. The composition according to claim 1, wherein the pregelatinized starch is prepared from a starch-containing plant source.

7. The composition according to claim 1, wherein the amount of pregelatinized starch ranges from 10% to 90% by weight relative to the total weight of the composition.

8. The composition according to claim 1, wherein the pregelatinized starch has a particle size distribution Dv(90) ranging from 90 to 1300 and/or a Dv(50) ranging from 40 to 500 and/or a Dv(10) ranging from 10 to 150.

9. The composition according to claim 1, wherein the ratio [mass of calcium carbonate source]: [mass of pregelatinized starch] ranges from 0.4 to 5.7.

10. The composition of claim 1, wherein the microorganism is a microorganism of probiotic interest.

11. The composition of claim 1, wherein the microorganism is a bacterium, a fungus or a yeast.

12. The composition according to claim 1, wherein the microorganism is a probiotic bacterium selected from bacteria of the genera Lactobacillus spp., Bifidobacterium spp. and mixtures thereof.

13. The composition according to claim 1, wherein the number of microorganism ranges from 10³ to 10¹² cells/g of composition.

14. A method for preparing the composition according to claim 1, comprising the step of mixing a microorganism with a calcium carbonate source and pregelatinized starch.

15. A method for protecting a microorganism in an acidic environment, wherein mixture of pregelatinized starch and a calcium carbonate source is supplied to said for protecting a microorganism in said acidic environment.

16. The method according to claim 15, wherein the calcium carbonate source is selected from limestones (e.g., chalk), snail shells, eggshells, marine animal shells, corals and algae of the order Corallinales (e.g., lithothamnion), preferably the calcium carbonate source is lithothamnion.

17. The method according to claim 15, wherein the calcium carbonate source has a particle size distribution Dv(90) ranging from 29 μm to 750 μm and/or a Dv(50) ranging from 7 μm to 500 μm and/or a Dv(10) ranging from 1 μm to 270 μm.

18. The method according to claim 15, wherein the pregelatinized starch is prepared from a starch-containing plant source, preferably selected from pregelatinized corn starch, pregelatinized pea starch, pregelatinized potato starch, pregelatinized tapioca starch, pregelatinized rice starch, pregelatinized cassava starch, preferably pregelatinized corn starch or pregelatinized potato starch.

19. The method according to claim 15, wherein the pregelatinized starch has a particle size distribution Dv(90) ranging from 90 to 1300 and/or a Dv(50) ranging from 40 to 500 and/or a Dv(10) ranging from 10 to 150.

20. The method according to claim 15, wherein the ratio [mass of calcium carbonate source]: [mass of pregelatinized starch] ranges from 0.4 to 5.7, preferably from 0.8 to 1.9.

21. The method according to claim 150, wherein the microorganism is a microorganism of probiotic interest.

22. The method according to claim 15, wherein the microorganism is a bacterium, fungus or yeast.

23. The method according to claim 15, wherein the microorganism is a probiotic bacterium selected from bacteria of the genera Lactobacillus spp., Bifidobacterium spp. and mixtures thereof.

24. The composition according to claim 1, wherein the water activity (a_{w}) is less than 0.06.

25. The composition of claim 1, wherein preferably the calcium carbonate source is lithothamnion.

26. The composition according to claim 1, wherein the pregelatinized starch is prepared from a starch-containing plant source selected from pregelatinized corn starch, pregelatinized pea starch, pregelatinized potato starch, pregelatinized tapioca starch, pregelatinized rice starch, pregelatinized cassava starch.

27. The composition according to claim 1, wherein the pregelatinized starch is prepared from a starch-containing plant source selected from pregelatinized corn starch or pregelatinized potato starch.

28. The composition according to claim 1, wherein the ratio [mass of calcium carbonate source]: [mass of pregelatinized starch] ranges from 0.8 to 1.9.

29. The composition according to claim 1, wherein the number of microorganism ranges from 10⁵ to 10¹¹ cells/g of composition.

30. The use method according to claim 15, wherein the calcium carbonate source is lithothamnion.

31. The method according to claim 15, wherein the ratio [mass of calcium carbonate source]: [mass of pregelatinized starch] ranges from 0.8 to 1.9.
